(19) United States
(12) Patent Application Publication
(10) Pub. No.: US 2004/0038229 A1
(43) Pub. Date: Feb. 26, 2004

(54) Enzymatic manipulation of metal particle-bound DNA

(76) Inventors: Christine Dolan Keating, University Park, PA (US); Sheila R.N. Pena, University Park, PA (US); Glenn P. Goodrich, University Park, PA (US); Surabhi Raina, University Park, PA (US); Nina V. Fedoroff, University Park, PA (US)

Correspondence Address: MCKEE, VOORHEES & SEASE, P.L.C., ATTN: PENNSYLVANIA STATE UNIVERSITY, 801 GRAND AVENUE, SUITE 3200, DES MOINES, IA, 50309-2721, US

(21) Appl. No.: 10/286,024

(22) Filed: Nov. 1, 2002

Related U.S. Application Data
(60) Provisional application No. 60/335,151, filed on Nov. 1, 2001.

(51) Int. Cl. C12Q 1/68; C12P 19/34

(52) U.S. Cl. 435/6; 435/91.2

(57) ABSTRACT

The invention provides various methods for enzymatically manipulating nanoparticle-bound nucleic acids. Such methods include single-stranded primer extension, reverse transcription, minisequencing/single nucleotide polymorphism detection or minisequencing, polymerase-based covalent immobilization of DNA.

### CROSS-REFERENCE TO RELATED APPLICATIONS

This application claims priority under 35 U.S.C. § 119(e) to to U.S. Provisional Patent Application No. 60/335,151, filed Nov. 1, 2001, the disclsoure of which is herein incorporated by reference.

### GRANT REFERENCE

Work for this invention was funded in part by a grant from United States National Institutes of Health Grant No. R01 HG02228, and in part by a grant froom National Science Foundation Grant No. DBI 9872629. The United States Government may have certain rights in this invention.

### FIELD OF THE INVENTION

This invention relates generally to the fields of bioanalytical chemistry and nanotechnology. More specifically, this invention relates to the enzymatic manipulation of nanoparticle-bound DNA.

### BACKGROUND OF THE INVENTION

Nano- and microscopic particles have enormous potential as amplification and identification tags in biological analysis (Elghanian et al., Science 277:1078-81 (1997); Mirkin et al., Nature 382:607-9 (1996); Chan et al., Science 281:2016-8 (1998); Han et al., Nature Biotechnology 19:631-5 (2001); Nicewarner-Peña et al., Science 294:137-41 (2001); Ye et al., Human Mutation 17:305-16 (2001); Walt Science 287:451-2 (2000); Battersby et al., J. Am. Chem. Soc. 122:2138-9 (2000); Dunbar et al., Clin. Chem 46:1498-1500 (2000); Gerion et al., J. Phys. Chem. B. 105:8861-71 (2001); Taylor et al., Anal. Chem. 72:1979-86 (2000)). In particular, colloidal gold (Au) nanoparticles have been used as amplification tags in a variety of assay formats based on their high absorbance and scattering cross sections, high density, small size, monodispersity, ease of derivatization, and commercial availability. While protein:Au nanoparticle conjugates have been used for decades, and have found increasingly broad application in recent years (Lyon et al., Anal. Chem. 70:5177-83 (1998); Gu et al., Supremol. Sci. 5:695-8 (1998)), it is only recently that nucleic acids have been coupled to colloidal Au and shown to retain the ability to selectively and reversibly hybridize to complementary sequences. Mirkin, Letsinger and coworkers used 5′ thiol moieties to prepare DNA oligomer:Au nanoparticle conjugates, and have demonstrated a variety of Au nanoparticle based DNA assays in which absorbance, scattering, and even Ag plating were employed to improve sensitivity. Further amplification was possible by electroless Ag deposition onto Au nanoparticles after selective adsorption to a surface, resulting in 50 fM detection limits for DNA oligonucleotides (Taton et al., Science 289:1757-60 (2000)), while scattering has been used to image single nanoparticles (Taton et al., J. Am. Chem. Soc. 123:5164-5 (2001); Yguerabide et al., Anal. Biochem. 262:157-76 (1998)). DNA:Au conjugates have also been used to improve detection limits for DNA in an imaging surface plasmon resonance assay (He et al., J. Am. Chem. Soc. 122:9071-7 (2000)).

In addition to applications in ultrasensitive detection, DNA:Au conjugates have been employed as building blocks for “bottom-up” assembly strategies. Alivisatos and coworkers demonstrated that several nanoscale Au building blocks could be positioned with high accuracy by attaching them to a single long strand of DNA (Alivisatos et al., Nature 382:60911 (1996)). Niemeyers et al. have synthesized DNA-strepavidin networks that served as scaffolding for the assembly of 1.4-nm Au nanocrystals (Niemeyer et al., Angew. Chem. Int. Ed. 37:2265-8 (1998)). Larger DNA-nanoparticle assemblies have been constructed in which two different nanoscale building blocks are alternated based on selective DNA hybridization and in which particle multilayers are built up on a glass substrate via consecutive hybridizations (Mucic et al., J. Am. Chem. Soc. 120:12674-5 (1998). Recently, DNA hybridization has been used to assemble Au nanoparticles onto patterned substrates via a lithographic approach (Moller et al., Nucleic Acids Res. 28:e91 (2000)) and by dip-pen nanolithography (Demers et al., Angew. Chem. Int. Ed. 40:3071-3 (2001)). DNA complementarity has also been used to direct the assembly of Au wires several hundred nm in diameter and several microns long onto planar Au surfaces (Martin et al., Advanced Materials 11:1021-5 (1999)).

Despite recent research activity in DNA:Au conjugates, no reports have been made of enzymatic manipulation of Au nanoparticle-bound DNA. In contrast, DNA bound to a variety of planar surfaces has been used in ligation, extension, and restriction endonuclease reactions (Syvanen, Human Mutation 3:172-9 (1994); Pirrung et al., J. Am. Chem. Soc. 122:187382, (2000); Pirrung et al., Langmuir 16:2185-91 (2000); Nilsson et al., Anal. Biochem. 224:400-8 (1995); Pastinen et al., Clin. Chem. 42:1391-97 (1996); Pastinen et al., Genome Research 7:606-14 (1997); Nikiforov et al., Nucleic Acids Research 22:4167-75 (1994); Braun et al., Clin. Chem. 43:1151-58 (1997)).

Polymer and glass bead-bound DNA has been extended, ligated, enzymatically cleaved, and, recently, PCR amplified (Andreadis et al., Nucleic Acids Res. 28: e5 (2000); Hakala et al., Bioconj. Chem. 8:378-84 (1997); Hakala et al., Bioconj. Chem. 8:232-7 (1997); Kwiatkowski, Nucleic Acids Res. 27:4710-4 (1999); Ordoukhanian et al., Nucleic Acids Res. 25:3783-6 (1997); Shumaker et al., Human Mutation 7:34654 (1996); Tully et al., Genomics 34:107-13 (1996)). Adaption of these enzymatic processing protocols for use on Au nanoparticles would significantly increase the toolkit available for DNA:nanoparticle applications ranging from sensing to materials assembly. For example, extension would enable the sequence of a short primer, oligonucleotide bound to an Au particle to be covalently modified for complementarity to any desired template strand. This would allow preparation of DNA:Au conjugates with the high overall coverage of DNA oligomers optimal for conjugate stability (Demers et al., Anal. Chem. 72:5535-41 (2000)) while controlling the number of long DNA strands presented to solution.

### BRIEF SUMMARY OF THE INVENTION

In one aspect, the invention provides a method for extending a nucleic acid bound to a nanoparticle comprising binding to a nanoparticle a single-stranded DNA primer; annealing to the nanoparticle-bound primer a single-stranded DNA; and enzymatically extending the primer.

In another aspect, the invention provides a method for reverse transcribing mRNA directly onto a nanoparticle comprising binding to a nanoparticle a single-stranded DNA primer; annealing to the nanoparticle-bound primer a single-stranded mRNA; and reverse transcribing the mRNA.

In another aspect, the invention provides a method for determining the identity of a specific nucleotide at a defined site in a nucleic acid comprising binding to a nanoparticle a single-stranded DNA primer via its 5′ end; annealing to the nanoparticle-bound primer a single-stranded DNA having a specific nucleotide whose identity is to be determined such that the 3′ end of the primer binds to a nucleotide flanking the specific nucleotide whose identity is to be determined; subjecting the nanoparticle-bound primer and annealed DNA to a polymerizing agent in a mixture containing each of ddATP, ddGTP, ddCTP, and ddTTP, wherein each of ddATP, ddGTP, ddCTP, and ddTTP are labeled with a different label, such that the primer is extended by a single nucleotide; and detecting the identity of the single nucleotide added to the 3′ end of the primer.

In another aspect, the invention provides a method for introducing sidedness to a nanoparticle comprising binding to a nanoparticle a plurality of first single-stranded DNA molecules; binding to a solid support a plurality of second single-stranded DNA molecules, wherein the first and second single-stranded DNA molecules are complementary to each other; contacting the nanoparticle with the solid support such that those first single-stranded DNA molecules nearest the solid support anneal to the second single-stranded DNA molecules contained thereon, and those first single-stranded DNA molecules furthest from the solid support do not anneal to the second single-stranded DNA molecules contained thereon and thus remain free, resulting in a nanoparticle having first single-stranded DNA molecules that are unannealed and free, and first single-stranded DNA molecules that are annealed and not free; subjecting the nanoparticle to an agent that modifies those first single-stranded DNA molecules that are unannealed and free, but does not modify those first single-stranded DNA molecules that are annealed and not free; and separating the nanoparticle from the solid support, thereby resulting in a nanoparticle having first and second sides, wherein the first side contains modified first single-stranded DNA molecules, and wherein the second side contains unmodified first single-stranded DNA molecules.

In another aspect, the invention provides a method for generating covalently immobilized DNA comprising binding a first single-stranded DNA primer to a nanoparticle; mixing the nanoparticle with a DNA having first and second complementary strands under conditions such that the first complementary strand of the DNA anneals to the nanoparticle-bound primer; and enzymatically extending the first primer.

### BRIEF DESCRIPTION OF THE DRAWINGS

FIG. 1. Surface coverage as a function of solution mole ratio for primers C₆P12 (▪), C₁₂P12 (●), and C₆N7P12 (♥) diluted with C₆A6 and adsorbed to 12-nm diameter Au nanoparticles.

FIG. 2. Effect of template length and primer coverage on hybridization efficiency with three primer to template ratios-excess, 5:1 and 10:1. C₆P12:Au conjugates were hybridized with complementary (template) oligos T12F (▪) and T88F (□). Dashed line ( - - - ) represents 100% hybridization efficiency. Hybridization was quantitated via fluorescence of bound T12F or T88F after removal from the particles (see text for details). As a control, a non-complementary oligo was used, N12Fc, for which the fluorescence measurement was below background.

FIG. 3. Effect of linker length and primer coverage on hybridization efficiency at a primer to template ratio of 10:1. C₆P12:Au (▪), C₁₂P12:Au (●), and C₆N7P12:Au (♥) conjugates were hybridized with complementary (template) oligos T12F (closed symbols) and T88F (open symbols). Dashed line ( - - - ) represents 100% hybridization efficiency. Hybridization was quantitated via fluorescence of bound T12F or T88F after removal from the particles (see text for details). As a control, a non-complementary oligo was used, N12Fc, for which the fluorescence measurement was below background.

FIG. 4. 4.0% Metaphor® agarose gel (A and B) and a 15% polyacrylamide denaturing gel (C) of reactions 1-10 in Table II. The template (T88) was run in lane (T) for internal orientation and comparison to the extended products. Evidence for incorporation of the fluorescently labeled Alexa dUTP is shown in (A), in which this is the gel prior to staining with Ethidium bromide. The same gel after staining is shown in (B). Note that the products in lanes 3-6 and 10 are brighter due to the enhanced fluorescence from the Alexa dUTP. The agarose gel was run in 0.5×TBE for 4 hours at 3.0 V/cm. and the acrylamide gel was run in 0.5×TBE for 1 hour at 200 V/hr.

FIG. 5. 4.0% Metaphor® agarose gel (A and B) and a 15% polyacrylamide denaturing gel (C) of reactions 1-16 in Table III. The template (T88) was run in lane (T) for internal orientation and comparison to the extended products. Evidence for incorporation of the fluorescently labeled Alexa dUTP is shown in (A), in which this is the gel prior to staining with Ethidium bromide. The same gel after staining is shown in (B). Note that the products in lanes 1 and 5-13 are brighter due to additional fluorescence from the Alexa dUTP. The agarose gel was run in 0.5×TBE for 4 hours at 3.0 V/cm, and the acrylamide gel was run in 1×TBE for 50 minutes at 200 V/hr.

FIG. 6. Comparison of enzymatic efficiency on differing linker and primer lengths as well as primer surface coverage of particle-bound primers. Extension was achieved using T88 as the template and Klenow for enzymatic for 2 hours at 37° C. Quantitation of incorporated nucleotides was determined via Alexa Fluor® 488-5-dUTP using a fluorimeter.

FIG. 7. 3.0% nondenaturing agarose gel of DNA:Au conjugates used in reactions 3-16 in Table III. The gel shows conjugates both before (B) and after (A) extension. Conjugates run in lanes labeled S were made using C₆A6 which was used as a standard.

FIG. 8. Graphical representation of introducing sidenedness to a nanoparticle.

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The practice of the present invention will employ, unless otherwise indicated, conventional techniques of molecular biology, chemistry, biochemistry and recombinant DNA technology, which are within the skill of the art. Such techniques are explained fully in the literature. See, e.g., MOLECULAR CLONING: A LABORATORY MANUAL, 3^{rd} ed., Sambrook et al., Cold Spring Harbor Laboratory Press, Cold Spring Harbor, N.Y. (2001); DNA CLONING, vols. I and II, Glover, ed. (1985); OLIGONUCLEOTIDE SYNTHESIS, Gait, ed. (1984); NUCLEIC ACID HYBRIDIZATION, Hames and Higgins, eds. (1984); and the series METHODS IN ENZYMOLOGY, Colowick and Kaplan, eds., Academic Press, Inc., San Diego, Calif.

Units, prefixes, and symbols may be denoted in their SI accepted form. Unless otherwise indicated, nucleic acids are written left to right in 5′ to 3′ orientation. Numeric ranges are inclusive of the numbers defining the range. Nucleotides may be referred to by their commonly accepted single-letter codes. The terms defined below are more fully defined by reference to the specification as a whole.

As used herein, the term “nanoparticle” is intended to refer to a material comprising, for example, colloidal metals, including, but not limited to, gold, silver, copper, nickel, cobalt, rhodium, palladium, platinum, etc, and any combination thereof, semiconductor materials including, but not limited to, CdS, CdSe, CdTe, Si, etc., and/or magnetic colloidal materials (i.e. ferrogmagnetite). Methods of making colloidal metal particles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991). Suitable metal particles are also commercially available from, e.g., Ted Pella, Inc. (gold), Amersham Corporation (gold) and Nanoprobes, Inc. (gold). The term “nanoparticle” is also intended to encompass cylindrical wires, referred to herein as “nanowires,” comprising, for example, any of these materials along the legnth of the wire. Such “nanowires” are described in, for example, Nicewarner-Peña et al., Science 294:137-41 (2001); Mbindyo et al., Adv. Mater. 13:249-54 (2001); and Peña et al., J. Phys. Chem. B 106:7458-62 (2002). The wire may comprise a single material, or several materials, preferably in the form of segments, resulting in a “striped” wire. For colloidal particles, the particle can range in size from about 1 nm to about 150 nm in diameter, more preferably from about 5 nm to about 100 nm in diamter, and even more preferably from about 10 nm to about 50 nm in diamter. For cylindrical nanowires, the length of the wire is from about 10 nm to about 10 μm or greater in length, and from about 1 nm to about 10 μm in width. Other nanoparticles include

As used herein, the term “primer” is intended to refer to a short (i.e. between 10-100 bases), single-stranded DNA or RNA that is capable of hybridizing to another single-stranded nucleic acid molecule, and which serves as a platform for the initiation of polynucleotide synthesis.

As used herein, “nucleic acid” includes reference to a deoxyribonucleotide or ribonucleotide polymer (DNA or RNA) in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

As used herein, “mRNA” or “messenger RNA” is intended to refer to the class of RNA molecules that copies the genetic information from DNA, in the nucleus of a cell, and carries it to ribosomes, in the cytoplasm, where it is translated into protein. mRNA contain, at their 3′ end, a series of adenine residues, referred to as a “poly-A tail.”

As used herein, “cDNA” or “complementary DNA” is intended to refer to DNA synthesized from an RNA template using reverse transcriptase.

As used herein, “polynucleotide” is intended to refer to a polymer of nucleotides and includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

As used herein, the term “anneal” and its derivatives is intended to refer to the action of contacting complementary RNA or DNA sequences with each other such that they become chemically bound to each other via hydrogen bonding. As used herein, the term “complementary,” with respect to DNA or RNA, is intended to refer to the matching strand of a DNA or RNA molecule to which its bases pair. Adenine pairs with thymine and uracil, and guanine pairs with cytosine.

As used herein, the term “reverse transcribing” is intended to refer to the making of a cDNA from a mRNA template via the enzyme reverse transcriptase and the four deoxyribonucleotide triphosphates (dNTPs). Reverse transcriptase requires a single-stranded DNA primer for initiating cDNA synthesis.

As used herein, “dideoxyribonucleoside triphosphate” or “ddNTP,” where “N” represents one of adenine (A), guanine (G), cytosine (C), thymine (T), or uracil (U), is intended to refer to a deoxyribonucleoside that lacks a 3′ hydroxyl group, and is thus unable to form a 3′-5′ phosphodiester bond necessary for chain elongation.

Extension of Nanoparticle-Bound DNA

In a first aspect, the invention provides a method for extending a nucleic acid bound to a nanoparticle comprising binding to a nanoparticle a single-stranded DNA primer; annealing to the nanoparticle-bound primer a single-stranded DNA; and enzymatically extending the primer.

The primer can be bound to the nanoparticle by any suitable method, including, for example, via covalent attachment, direct adsorption, or noncovalent molecular recognition interactions. Specific examples include coating the nanoparticle with avidin (i.e. streptavidin, neutravidin) followed by exposure of the nanoparticle to biotinylated primers, covalent coupling of aminated primers to carboxyl-terminated self-assembled alkanethiols, and direct adsorption of thiolated primers (Mbindyo et al., Advanced Materials 13:249-54 (2001); Reiss et al., MRS Symp. Proc. 635:C6.2.16.2.6 (2001)). Preferably, the primer is bound to the nanoparticle via a 5′ thiol linker. The linker can comprise CH₂ moieties, as well as additional nucleotides.

Enzymatic extension of the primer can be accomplished by any suitable method currently known or developed in the future. Such methods are described in, for example, MOLECULAR CLONING: A LABORATORY MANUAL, 3^{rd} ed., Sambrook et al., Cold Spring Harbor Laboratory Press, Cold Spring Harbor, N.Y. (2001). Preferably, the primer is extended at its 3′ end by the addition of the four deoxyribonucleotide triphosphates (dNTPs) in the presence of a DNA polymerase such as, for example, the Klenow fragment of E. coli DNA polymerase.

Reverse Transcription of mRNA into DNA on Nanoparticles

In another aspect, the invention provides a method for reverse transcribing mRNA directly onto a nanoparticle comprising binding to a nanoparticle a single-stranded DNA primer; annealing to the nanoparticle-bound primer a single-stranded mRNA; and reverse transcribing the mRNA.

Preferably, the primer is a poly-dT primer. The primer can be bound to the nanoparticle by any suitable method, including, for example, via covalent attachment, direct adsorption, or noncovalent molecular recognition interactions. Specific examples include coating the nanoparticle with avidin (i.e. streptavidin, neutravidin) followed by exposure of the nanoparticle to biotinylated primers, covalent coupling of aminated primers to carboxyl-terminated self-assembled alkanethiols, and direct adsorption of thiolated primers (Mbindyo et al., Advanced Materials 13:249-54 (2001); Reiss et al., MRS Symp. Proc. 635:C6.2.16.2.6 (2001)). Preferably, the primer is bound to the nanoparticle via a 5′ thiol linker. The linker can comprise CH₂ moieties, as well as additional nucleotides.

Methods of reverse transcribing mRNA into cDNA are well-known in the art, and are described in, for example, MOLECULAR CLONING: A LABORATORY AL, 3^{rd} ed., Sambrook et al., Cold Spring Harbor Laboratory Press, Cold Spring Harbor, N.Y. (2001).

According to the method, once reverse transcription of mRNA directly onto the nanoparticle has been accomplished, the cDNAs will be intrinsically tagged with nanoparticles that can then be used as amplification tags or identifiable supports. Such tags could be used to increase the sensitivity of detection mechanisms that rely on cDNA binding to its complement on a solid support. This approach makes it possible to use particle-amplified detection schemes (particle-amplified surface plasmon resonance, scattering, absorbance, scanning probe microscopies, electron microscopies, surface enhanced vibrational spectroscopies, etc.) without resorting to additional hybridization steps, and is compatible with standard microarray synthesis and hybridization methods.

Minisequencing/Single Nucleotide Polymorphism Detection

In another aspect, the invention provides a method for determining the identity of a specific nucleotide at a defined site in a nucleic acid comprising binding to a nanoparticle a single-stranded DNA primer via its 5′ end; annealing to the nanoparticle-bound primer a single-stranded DNA having a specific nucleotide whose identity is to be determined such that the 3′ end of the primer binds to a nucleotide flanking the specific nucleotide whose identity is to be determined; subjecting the nanoparticle-bound primer and annealed DNA to a polymerizing agent in a mixture containing each of ddATP, ddGTP, ddCTP, and ddTTP, wherein each of ddATP, ddGTP, ddCTP, and ddTTP are labeled with a different label, such that the primer is extended by a single nucleotide; and detecting the identity of the single nucleotide added to the 3′ end of the primer.

Since this method involves the addition to the primer of a single, chain-terminating ddNTP, in addition to the method wherein each of the four ddNTPs are labeled and included in the mixture, the invention also encompasses methods wherein the mixture contains at least one labeled ddNTP, either alone or in combination with any other labeled or unlabeled ddNTP.

The primer can be bound to the nanoparticle by any suitable method, including, for example, via covalent attachment, direct adsorption, or noncovalent molecular recognition interactions. Specific examples include coating the nanoparticle with avidin (i.e. streptavidin, neutravidin) followed by exposure of the nanoparticle to biotinylated primers, covalent coupling of aminated primers to carboxyl-terminated self-assembled alkanethiols, and direct adsorption of thiolated primers (Mbindyo et al., Advanced Materials 13:249-54 (2001); Reiss et al., MRS Symp. Proc. 635:C6.2.16.2.6 (2001)). Preferably, the primer is bound to the nanoparticle via a thiol linker. The linker can comprise CH₂ moieties, as well as additional nucleotides.

According to this method, since ddNTPs are used, the primer is extended by only a single nucleotide.

Preferably, the different labels used to label each ddNTP are spectrally-distinct fluorescent labels. Particularly preferred spectrally-distinct fluorescent labels include Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700 and Alexa Fluor® 750 dyes. Other suitable spectrally-distinct fluorescent labels include fluorescein, rhodamine, Cy3, Cy5, Cy5.5, Cy7, etc. If a single ddNTP is used, then it is not necessary to use a fluorescent label, but can be, for example, a radioactive label.

According to the method, detection of the specific nucleotide added to the 3′ end of the primer will depend upon the labels that are used to label each ddNTP. If, for example, each ddNTP is labeled with a different fluorescent label, as indicated, then detection of the nucleotide can be acocomplished by any suitable fluorescence detection method.

The polymerizing agent is any enzyme capable of primer-dependent extension of nucleic acids. Preferably, the enzyme is a DNA polymerase such as, for example, Klenow, T7 DNA polymerase, and T4 DNA polymerase. Thermostable DNA polymerases can also be used in this method.

It is possible to extend the method to a multiplexed format. Any encoded nanoparticle could be employed as the support. One such particle is a barcoded nanowire. See, e.g., Nicewarner-Peña et al., Science 294:137-41 (2001).

Introduction of Sidedness to Nanoparticles

In another aspect, the invention provides a method for introducing sidedness to a nanoparticle comprising binding to a nanoparticle a plurality of nucleic acid molecules; contacting the nanoparticle with the solid support; and subjecting the nanoparticle to an agent that modifies those nucleic acid molecules furthest from the solid support, but does not modify those nucleic acid molecules closest to the solid support, thereby resulting in a nanoparticle having first and second sides.

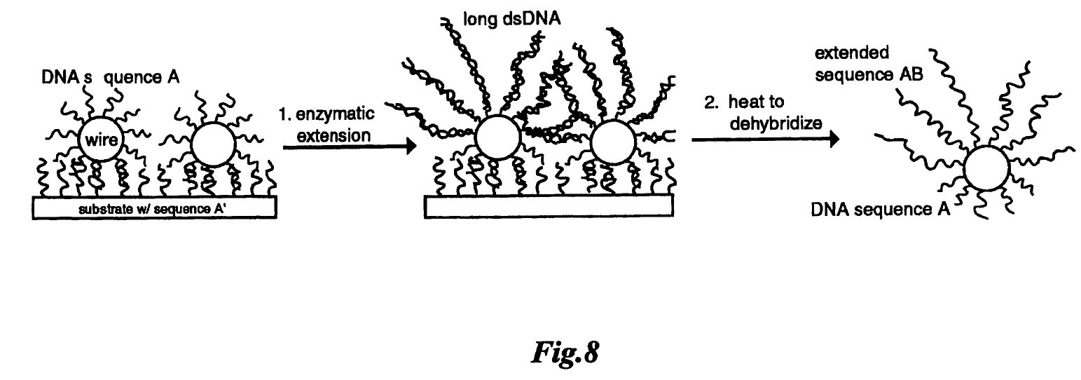

In a preferred embodiment, the method comprises binding to a nanoparticle a plurality of nucleic acid molecules; binding to a solid support a plurality of second nucleic acid molecules, wherein the first and second nucleic acid molecules are complementary to each other; contacting the nanoparticle with the solid support such that those first nucleic acid molecules nearest the solid support anneal to the second nucleic acid molecules contained thereon, and those first nucleic acid molecules furthest from the solid support do not anneal to the second nucleic acid molecules contained thereon and thus remain free, resulting in a nanoparticle having first nucleic acid molecules that are unannealed and free, and first nucleic molecules that are annealed and not free; subjecting the nanoparticle to an agent that modifies those first nucleic acid molecules that are unannealed and free, but does not modify those first nucleic acid molecules that are annealed and not free; and separating the nanoparticle from the solid support, thereby resulting in a nanoparticle having first and second sides, wherein the first side contains modified first nucleic acid molecules, and wherein the second side contains unmodified first nucleic acid molecules. In this embodiment, the nucleic acid molecules can be DNA or RNA. This embodiment is exemplified in FIG. 8.

The plurality of nucleic acid molecules can be bound to the nanoparticle by any suitable method, including, for example, via covalent attachment, direct adsorption, or noncovalent molecular recognition interactions. Specific examples include coating the particle with avidin (i.e. streptavidin, neutravidin) followed by exposure of the nanoparticle to biotinylated nucleic acid, covalent coupling of aminated nucleic acid to carboxyl-terminated self-assembled alkanethiols, and direct adsorption of thiolated DNA (Mbindyo et al., Advanced Materials 13:249-54 (2001); Reiss et al., MRS Symp. Proc. 635:C6.2.1-6.2.6 (2001)). The nucleic acid molecules can be bound to the nanoparticle via either their 5′ or 3′ ends.

According to the method, the solid support can be, for example, a microwell plate, a tube, a bead, a glass slide, a silicon wafer, or a membrane.

According to the method, once a nanoparticle is obtained having first nucleic acid molecules that are unannealed and free, and first nucleic acid molecules that are annealed (to theeir complements on the solid support), the nanoparticle is subjected to an agent that modifies those first nucleic acid molecules that are unannealed and free, but does not modify those first nucleic acid molecules that are annealed and not free. Suitable agents include enzymes such as polymerases (i.e. DNA polymerase), ligases, kinases, nucleases, and phosphatases, and RNAses.

Once the nanoparticle is subjected to an agent that modifies those first nucleic acid molecules that are unannealed and free, but does not modify those first nucleic acid molecules that are annealed and not free, the nanoparticle is separated from the solid support. Such separation can be accomplished thermally or chemically.

For nanoparticles formulated into a nanowire, the method could result in different nucleic acid molecules on two parts along the length of the wire, in contrast to any previous orthogonal derivatization strategies. Current methods of DNA-directed assembly (indeed, nearly any type of particle self-assembly) employ nano- and micro-particles with uniform chemistries over their entire surface. One notable exception is when orthogonal derivatization of Au and Pt segments of a single metal nanowire is used to place DNA on e.g., only the Au segments (Martin et al., Advanced Materials 11:1021-25 (1999)). This allows different DNA sequences to be placed in different locations on the nanowire. However, the nanowire remains symmetrically derivatized around its long axis. This symmetry limits the number of possible nanowire connections that can be selected for via DNA-directed assembly. For example, in a two-dimensional “raft” of parallel nanowires, only palindromic nanowire sequences can be prepared. Chemistry to remove the symmetry around the nanowire long axis could generate a “dorsal” and “ventral” sidedness and enable two-dimensional rafts to be prepared in any desired order. The combination of Au/Pt orthogonal chemistry and this sidedness together could make possible a wide range of complex, deterministic, DNA-directed assemblies that may find application in self-assembly nanoscale circuitry.

This method also encompasses situations where the solid support contains no nucleic acid molecules, and the nanoparticles are simply gravitationally contacted to the solid support such that the nucleic acid molecule modifying agent cannot “reach” the molecules on the nanoparticle closest to the solid support.

It is also possible to introduce sidedness to nanoparticles without using a solid support. For example, the nanoparticles with the first nucleic acid molecules can be placed in one phase of a two-phase, aqueous solution, and the second nucleic acid molecules can be placed in the other phase of the solution. An example of such a solution is a polyethylene glycol:dextran solution.

Generation of Covalently Immobilized DNA

In another aspect, the invention provides a method for generating covalently immobilized DNA comprising binding a first single-stranded DNA primer to a nanoparticle; mixing the nanoparticle with a DNA having first and second complementary strands under conditions such that the first complementary strand of the DNA anneals to the nanoparticle-bound primer; and enzymatically extending the first primer.

Preferably, in the mixing step according to the method, a second single-stranded DNA primer is mixed with the nanoparticle and the DNA under conditions such that the second complementary strand of the DNA anneals to the second primer, and in the extending step, the second primer is enzymatically extended. Preferably, the mixing and extending steps are repeated one or more times.

The primer can be bound to the nanoparticle by any suitable method, including, for example, via covalent attachment, direct adsorption, or noncovalent molecular recognition interactions. Specific examples include coating the particle with avidin (i.e. streptavidin, neutravidin) followed by exposure of the particle to biotinylated primers, covalent coupling of aminated primers to carboxyl-terminated self-assembled alkanethiols, and direct adsorption of thiolated primers (Mbindyo et al., Advanced Materials 13:24954 (2001); Reiss et al., MRS Symp. Proc. 635:C6.2.1-6.2.6 (2001)). Preferably, the primer is bound to the nanoparticle via a 5′ thiol linker. The linker can comprise CH₂ moieties, as well as additional nucleotides.

Enzymatic extension of the primers can be accomplished by any suitable method currently known or developed in the future. Such methods are described in, for example, MOLECULAR CLONING: A LABORATORY MANUAL, 3^{rd} ed., Sambrook et al., Cold Spring Harbor Laboratory Press, Cold Spring Harbor, N.Y. (2001). Preferably, the primers are extended at their 3′ end by the addition of the four deoxyribonucleotide triphosphates (dNTPs) in the presence of a DNA polymerase such as, for example, Klenow, T7 DNA polymerase, T4 DNA polymerase, and most preferably, a thermostable DNA polymerase.

For primers attached to barcoded nanowires (see, e.g., Nicewarner-Peña et al., Science 294:137-41 (2001)), the reaction results in the sequence of interest on a readily identifiable support. The DNA sequence can then be read out via the particle “code” (i.e, when different metals are used). Detection of extension can be done via standard fluorescence-based methods or other means, and does not need to identify anything other than the fact that DNA has been extended on that particle. For example, fluorescent nucleotides could be employed, such that any DNA extended from a particle-bound primer fluoresced. This would enable instant detection of those nanoparticles for which the target sequence was present in a sample; nanoparticles could subsequently be read out via, e.g., a “barcode” pattern, a fluorescence signature, etc.

According to the method of the invention, amplicons bound to nanoparticles can be detected in situ via nanoparticle-amplified surface plasmon resonance, light scattering, or a variety of other methods, and amplicons bound to nanoparticles can be detected ex situ via the methods mentioned above, or other methods including scanometric DNA detection methods, gel electrophoresis, quartz-crystal microbalance, electrochemistry, or any other method which can detect the strong nanoparticle signal and distinguisyh primer-bound from amplicon-bound particles.

This method could be extended to a multiplexed format, in which many primers are present, each bound to a separate, encoded nanoparticle. Encoded nanoparticles could include metallic barcoded nanowires or fluorescently-encoded microbeads. Specific nanoparticle-bound primers could be added in proportion to the expected yield of amplicons in order to keep all of the amplicon detection events within the same dynamic range.

This invention can be better understood by reference to the following non-limiting example. It will be appreciated by those skilled in the art that other embodiments of the invention may be practiced without departing from the spirit and the scope of the invention as herein disclosed and claimed.

### EXAMPLE 1

Enzymatic Extension of Au Nanoparticle-bound Primers

In these experiments, a nanoparticle-bound 12-mer primer sequence is hybridized to an 88-mer template sequence. Addition of DNA polymerase leads to the covalent incorporation of nucleotides to form the complement of the template. Primers were attached via 5′ C₆H₁₂SH, C₁₂H₂₄SH, and TAACATTC₆H₁₂SH linkers. Prime coverage on the nanoparticles was varied by dilution with a 6-mer polyA oligonucleotide. Because hybridization is a prerequisite for extension, hybridization efficiencies were determined as a function of primer coverage, template length (12-mer vs. 88-mer), and primer:template concentration ratio. In all cases, hybridization for the 88-mer was less efficient than for the 12-mer. In the presence of excess template low primer coverages led to optimal hybridization. However, at the 10:1 primer:template ratio used for extension, hybridization efficiency did not depend strongly on surface coverage. In contrast, extension efficiency was significantly impacted by both parameters. Extension was observed via gel electrophoresis of DNA after removal from Au nanoparticles, and via fluorescence of incorporated dye-labeled dUTP. Nondenaturing gel electrophoresis of the DNA-coated nanoparticles was used to verify that extension occurred on the particles.

Enzymatic manipulation of DNA bound to metal nanoparticles presents some challenges not present for DNA on plastic, glass or microbeads. For example, the Au—S bond, although thermodynamically stable, is kinetically labile, leading to thiol exchange in the presence of thiol-containing molecules in solution, particularly at elevated temperatures. Buffers used in molecular biology often contain thiols, e.g., dithiothreitol, that are commonly included as reductants to prevent the formation of disulfide bonds in the enzymes. Note that it is possible to attach DNA to Au nanoparticles via avidin-biotin attachment chemistry, which would avoid the use of thiols altogether (Nicewarner-Peña et al., Science 294:137-41 (2001); Niemeyer et al., Angew. Chem. Int. Ed. 37:2265-68 (1998)). However, because thiol chemistry affords greater control over linker length and surface coverage, it is the method of choice. In addition, thiol-based linkers allow closer approach between Au particles and the surface to which they hybridize (e.g., another nanoparticle, a planar substrate) than do NA-biotin linkers; for detection mechanisms involving optical and electronic coupling, this greater separation can decrease sensitivity. Under the relatively mild reaction conditions for enzymatic extension (37° C., ˜1 μM DTT), no thiol exchange is observed.

Primer coverage and hybridization efficiencies were determined as a function of linker length and primer surface coverage via fluorescence of FITC-tagged oligos after removal from the particle surface. It was found that an 88-mer template DNA strand can be enzymatically extended under most conditions of linker and spacing, and that both the surface coverage and the linker length of the primers tested were important to enzymatic extension. Extension was followed via incorporation of fluorescently labeled dUTPs, and by gel electrophoresis of particle-bound and released DNA after extension.

The high efficiency with which nanoparticle-bound DNA could be extended was unexpected due to expected steric hindrance by the particle itself and other primer strands bound to the particle. However, by reducing the surface coverage of the primer and by increasing the length of the spacer between the primer sequence and the particle surface, efficiencies were achieved that were high as were observed for free, non-particle bound primers in solution.

Materials

H₂O used in all experiments was 18.2 MΩ, distilled through a Barnstead Nanopure system. HAuCl₄.3H₂O) was purchased from Acros. oligonucleotides used in this work were purchased from Integrated DNA technologies, Inc. (IDT) or the Nucleic Acid Facility (University Park campus). NaCl, NaH₂PO₄ were purchased from J.T. Baker Inc. Klenow (the large fragment of DNA polymerase I), React 2 buffer, and ultra pure agarose were purchased from Life Technologies. Alexa Fluor® 488-5-dUTP was purchased from Molecular Probes. Non-labeled dNTPs were purchased from Promega Life Sciences. Mercaptoethanol (MCE) and dithiothreitol (DTT) were purchased from Sigma. NAP-5 and NAP-10 columns were purchased from Amersham Pharmacia. TBE-Urea ready polyacrylamide gels, TBEUrea sample buffer, and Bio-Gel P-6-gel, medium grade was purchased from BioRad. MetaPhor® agarose was purchased from BioWhittaker Molecular Applications (Rockland, Me.).

Preparation of DNA:Au Conjugates

A list of all sequences used in this work is shown in Table I. Thiolated oligonucleotides used in this work were received as dithiols. The dithiol was cleaved using a 100 mM solution of DTT in 0.1 M Na phosphate pH 8.3 buffer. The reaction was allowed to proceed for 30 min at room temperature, after which the oligo was desalted on a NAP-5 or NAP-10 column with elution into autoclaved 18.2 MΩ H₂O. The purified solution of oligonucleotide was quantitated using A₂₆₀ and the extinction coefficient specific for the sequence. UV-vis spectra were acquired on a HP 8452A diode array ultraviolet-visible spectrophotometer with 2-nm resolution and 1-sec integration time. 12-nm diameter colloidal Au particles were prepared via citrate reduction of HAuCl₄ as previously described (Grabar et al., Anal. Chem. 69:471-77 (1997)). Particle size was determined by transmission electron micrographs (TEM) and NIH imaging software. [Imaging, #85] All conjugates used in this work were prepared using the same batch of colloidal Au particles, which were determined to be 13.1=1.3-nm×11.0=1.2-nm, further referred to as 12-nm throughout the paper.

DNA:Au conjugates were prepared similar to literature precedence with a few modifications (Storhoff et al., J. Am. Chem. Soc. 120:1959-64 (1998)). In short, 12.5 μL of a 100 μM solution of the oligonucleotide was added to 200 μL of the 12-nm colloidal Au sol. The final concentration of oligonucleotide and colloid was 5 μM and 13.1 nM, respectively. The samples were placed into a water bath at 37° C. for 8 hours, after which, the solution was brought to 0.1 M NaCl/10 mM Na phosphate (PBS) pH 7 at a total volume of 500 μL. The samples were left in the “aging” solution for at least 16 hours at 37° C. Following this, samples were centrifuged at 10,000 g for 40 minutes, twice, with a rinse of 500 μL in between. Samples were resuspended to a final volume 200 μL for analysis via fluorescence spectroscopy.

Preparation of conjugates for extension were made in the same fashion as stated above, except in this case, 60 μL of a 100 μM solution of the oligo was added to 1 mL of the colloidal Au sol. Surface diluted conjugates were prepared by addition of the primer and the diluent oligo C₆A6 in molar ratios indicated to yield a total oligo solution volume of 60 μL. Samples were centrifuged twice with a rinse of 1.5 mL between centrifugations. The samples were resuspended into 0.3 M PBS pH 7 at 350 μL (100% conjugates), −300 μL (50% conjugates), and ˜200 μL for (20% conjugates). 5 μL of each conjugate was taken for quantitation of primer concentration using values determined previously. The final volume of each conjugate varied so that the final concentration of primer was 3 μM. All DNA:Au conjugates used for extension were quality checked using a calorimetric solution assay as first described by Mirkin and coworkers (Elghanian et al., Science 277:1078-81 (1997)).

Fluorescence Quantitation of Primer Coverage on Au Particles

Primers used for these studes were labeled with 8carboxyfluorescein (6-FAM) at the 3′ end. Fluorescently labeled oligos were first adsorbed to the surface of 12-nm diameter colloidal Au particles following the protocol outlined above. For conjugates diluted with C₆A6, the primer diluent ratio indicates the ratio of primer to dilutor molecule present in the initial adsorption solution. For the surface diluted conjugates, only the primer oligo was fluorescently labeled. DNA:Au conjugates were washed and centrifuged twice to ensure removal of any non-specifically adsorbed molecules. The fluorescently labeled oligo was displaced using 12 mM mercaptoethanol (MCE) following established literature precedence (Demers et al., Anal. Chem. 72:5535-41 (2000)). The conjugates were placed into a 37° C. water bath and left for at least 8 hours. The conjugates were then centrifuged again at 10,000 g for 20 min, after which, the supernatant containing the fluorescently labeled oligo was removed and analyzed via fluorescence spectroscopy. Quantitation of fluorescently labeled oligonucleotides and incorporation of fluorescently labeled dUTPs was acquired on a SPEX Fluorolog model 1681 (0.22 m spectrometer) equipped with a PMT.

Hybridization Efficiency of DNA:Au Conjugates

DNA:Au conjugates were prepared as stated above and then resuspended into a final volume of 100 μL of 0.3 M PBS pH 7.0 μL from one of each conjugate dilution was removed for UV-vis analysis to determine the concentration of the primer. Conjugates were brought to a final volume of 200 μL for hybridization to 5′ 6-FAM fluorescently labeled complementary oligos T:12F and T88F. The samples were heated in a water bath to 65° C. for 5 minutes, removed and allowed to cool to room temperature for 30 minutes. The conjugates were heated again to 65° C. for 5 minutes and then allowed to anneal while cooling to room temperature for 120 minutes in the water bath. After annealing, the conjugates were centrifuged twice at 10,000 g for 40 min with washing of 500 μL between centrifugations. Conjugates were resuspended into a final volume of 200 μL and the solution pH was brought to 12 via addition of 45 μL of a 1.0 M NaOH solution to de-hybridize the bound oligonucleotide. The conjugates were placed onto a vortexer with gentle shaking for 2 hours. After 2 hours, the conjugates were centrifuged again at 10,000 g for 35 min. the supernatant was pH adjusted to 9 using ˜40 μL of a 1.0 M HCl solution (pH was confirmed using a pH test strip) and analyzed via fluorescence.

DNA Extension from Particle-Bound Primers

Conjugates used in extension reactions were prepared as stated above. Samples for control reactions in which the DNA:Au conjugate primer was noncomplementary to the template were prepared using C₆N18. A primer to template ratio of 10:1 was used in all experiments. In order to keep this ratio the same for conjugates that were surface diluted while maintaining the same amount of template molecules in each experiment, the concentration of Au particles for the surface diluted conjugates was increased such that the primer concentration was kept to 3 μM. Control reactions in which the primer was not attached to the Au particle were the same sequence except without modification (i.e. N12 and N18). For these reactions, 7.2 μL of a 10 μM solution of N12 or N18 was added to 7.2 μL of a 1 μM solution of N88. For samples in which the primer was attached to the Au particle, 24 μL of the conjugate was added to 7.2 μL of the template solution. The samples were brought to a final volume of 50 μL using 0.3 M PBS pH 7 for annealing. The reaction was heated to 65° C. for 5 minutes and allowed to cool to room temperature in the water bath for 2 hours. Following annealing of the template to the primer, the reactions were brought to a total reaction volume of 75 μL by addition of 7.5 μL 10× REact 2 buffer, nuclease free H₂₀, 1.1 μL of 50 μM Alexa dUTP, 4 μL of 250 μM dNTPs (150 μM dTTP), and 1 μL of 2U/×L of Klenow. The samples were placed into a water bath at 37° C. for 2 hours for extension. After the allotted time of 2 hours, the reaction was quenched by the addition of 4 μL of a 0.5 M EDTA pH 8.0 solution. Samples prepared to run on agarose gels in which the DNA remained on the Au particles were performed as stated above except in the following vectors. Only samples involving conjugates were used in this experiment. 48 μL of each conjugate was added to 14.4 μL of the template (T88) followed by the addition of 0.3 M PBS pH 7 to bring the total volume for annealing to 75 μL. The total volume for extension was brought to 100 μL by the addition of 10 μL 10× REact 2 buffer, nuclease free H₂O, 5 μM dNTPs, and 1 μL of 2U/μL Klenow.

After quenching the reaction, 1 μL of MCE was added to each sample and placed into a 37° C. water bath for 8 hours to aid the removal of the DNA from the Au particle. Samples were again centrifuged at 10,000 g for 15 minutes to bring all the aggregated particles to the bottom of the centrifuge tube. Prior to purification of samples via column chromatography to remove enzyme and unincorporated dNTPs, 15 μL was removed from each sample and saved for analysis on a MetaPhor® agarose gel. The remaining sample was purified via column chromatography using BioRad P-60 gel medium grade. The sample was applied to the column bed and allowed to migrate in. This was followed by 150 μL mobile phase (degassed 0.3 M PBS pH 7), during this time the eluent was sent to waste. Next, 450 μL of the mobile phase was added and the eluent was collected, which contained the dsDNA product. The amount of Alexa Fluor dUTP incorporated was determined via fluorescence spectroscopy. The samples were analyzed via fluorescence spectroscopy, with λₑₓ=493 nm and λₑₓ=515 nm. Standards of Alexa Fluor 488-5-dUTP were prepared ranging from 0.7 nM to 200 nM and run at the time of sample analysis. This was converted into the amount of dTTP incorporated based on the ratio of labeled dUTP to dTTP. From this, the total amount of nucleotides incorporated was calculated and the final amount of incorporated nucleotides, % nucleotides incorporated, was calculated based on the number of template molecules added to each reaction mixture. Agarose and polyacrylamide gels were imaged with AlphaImager™ 2200 documentation and analysis system equipped with a CCD and AlphaEase T image processing and analysis software. Agarose gels, in which the DAN remained on the particles, were scanned into a flatbed scanner and processed using Photoshop® version 5.0.

Results and Discussion

Steric factors are expected to play a role in extension efficiency both during hybridization of the template to particle-bound primers and during enzyme binding/extension. To distinguish between these two effects, hybridization efficiency as a function of coverage and linker length was first characterized.

Effect of Linker and Primer Surface Coverage on Hybridization Efficiencies

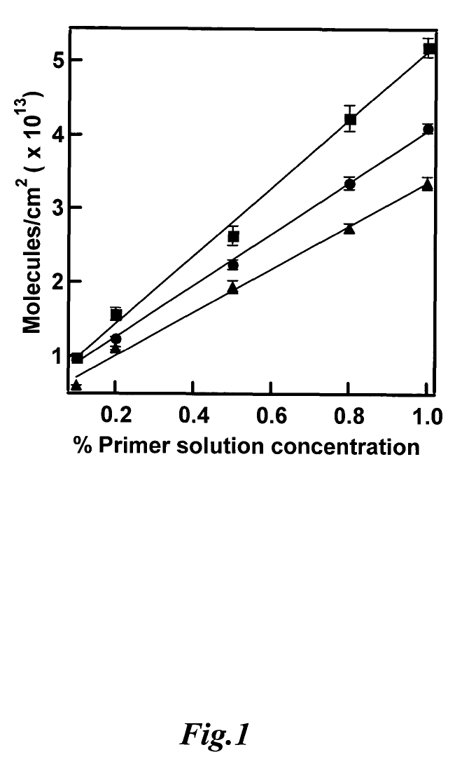

The oligonucleotides used to prepare DNA:Au conjugates in this study are of the form HS-linker-primer (see Table I for DNA sequences). Three different linkers (C₆H₁₂, C₁₂H₂₄, and C₆H₁₂N₇, abbreviated C₆, C₁₂, and C₆N7, respectively) were investigated between the 5′ thiol moiety and the primer sequence (P12). Primer coverage was controlled via competitive adsorption of specific primers (P12) with a nonspecific A6 oligonucleotide (HSC₆H₁₂AAA AAA). FIG. 1 reports the number of particle-bound specific primers for each linker at solution mole fractions ranging from 0.1 to 1.0. As expected, the C₆ linker gave the highest surface coverage of primers, with the longer linkers resulting in somewhat lower coverages in the order of their linker length. A second conclusion to be drawn from FIG. 1 is that primer coverage is directly proportional to solution mole fraction, in agreement with Demers et al. who report surface dilution of thiolated oligonucleotides with a 20-base polyA sequence on colloidal Au nanoparticles (Demers et al., Anal. Chem. 72:5535-41 (2000)). DNA:Au conjugates were prepared with primer coverages between 6.2×10¹² and 5.2×10¹⁵ molecules/cm² (28 and 234 molecules/particle) for investigation of primer coverage effects.

For enzymatic extension to occur, the particle-bound primer must first hybridize to the solution-phase template. The importance of both surface coverage and linker length in hybridization efficiency for surface-bound oligonucleotides has been demonstrated on planar surfaces and microbeads (Demers et al., Anal. Chem. 72:5535-41)2000); Steel et al., Biophys. J. 79:975-81 (2000); Shchepinov et al., Nucleic Acids Res. 25:1155-61 (1997); Southern et al., Nature Genetics Supp. 21:5-9 (1999); Herne et al., J. Am. Chem. Soc. 119:8916-20 (1997)). For example, Southern and coworkers found that the length of linker moieties, rather than their chemical makeup, was the most important parameter. They recommended linkers of 30 to 60 atoms between a planar substrate and the hybridizing DNA sequence (Southern et al., Nature Genetics Supp. 21:5-9 (1999)). It has also been demonstrated that decreased oligonucleotide surface coverage leads to improved hybridization efficiencies (Steel et al., Biophys. J. 79:975-81 (2000); Shchepinov et al., Nucleic Acids Res. 25:1155-61 (1997); Herne et al., J. Am. Chem. Soc. 119:8916-20 (1997); Levicky et al., J. Am. Chem. Soc. 120:9787-92 (1998)). Although the Au nanoparticles used in this work have a high radius of curvature, which is expected to reduce steric effects, it was hypothesized that these parameters would remain important for nanoparticle-bound DNA.

Indeed, Mirkin and coworkers have prepared DNA conjugates with 16-nm diameter colloidal Au nanoparticles, and observed improvements in hybridization efficiency from 4% to 44% with the addition of a 20 base nonhybridizing sequence between the nanoparticles and the 12-mer of interest (Demers et al., Anal. Chem. 72:5535-41 (2000)). The coverage for the longer sequence was substantially less than for the 12-mer, at 9.0×10¹² molecules/cm² as compared to 2.0×10¹³ molecules/cm² (Demers et al., Anal. Chem. 72:5535-41 (2000)). This was not unexpected; long DNA strands are known to result in lower surface coverages on planar substrates (Steel et al., Biophys. J. 79:975-98 (2000)). The linking sequences used in this work are much shorter, with the longest only C₆N7 or 49 atoms 2 nm). Thus, it was possible to achieve somewhat similar surface coverages with all three linkers, separating the effects of surface coverage and linker length. Maximum coverage for the three primer oligonucleotides ranged from 3.4-5.2×10¹³ molecules/cm² for these linkers.

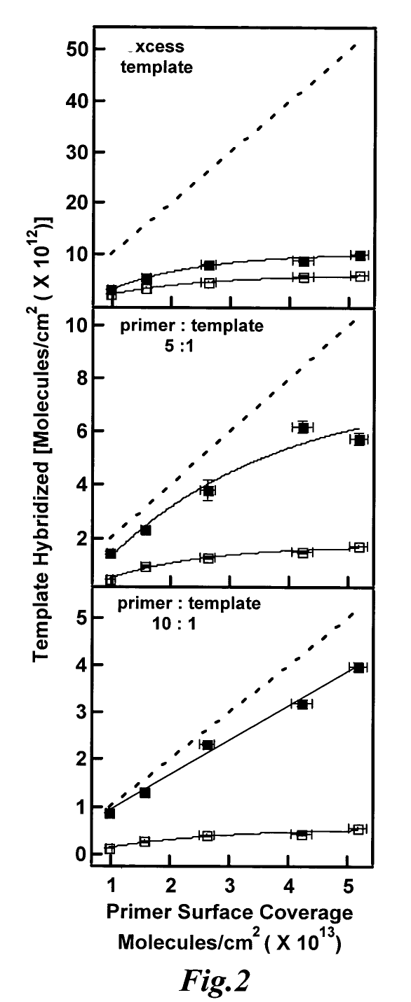

To determine the accessibility of surface-bound primers for hybridization, both 12-mer and 88-mer complements (these are referred to as “templates”; the 88-mer sequence is used as the template for extension in later experiments) were employed. FIG. 2 (left panel) shows the results of hybridization of particle-bound primers (C₆P12) with excess template (T12F and T88F) as a function of primer coverage. A dashed line illustrates the hybridized strans/cm2 expected if every primer binds a complementary strand from solution. Clearly, these data represent a much lower hybridization efficiency than 100%. Hybridization efficiencies are higher for the 12-mer sequence as compred to the 88-mer. This is consistent with the greater steric effects expected for the longer sequence. At high primer coverages, this difference is most significant. A maximum of −46 or −26 hybridization events occurred per particle for the 12-mer and 88-mer, respectively, corresponding to 20% and 11% of the −234 total primers on the particles. At the highest primer coverages, no more than 20% of primers were hybridized despite an excess of template in solution. However, the hybridization efficiency rose to −33% and 22% at low primer coverages.

For enzymatic extension, it is desirable to employ a template concentration less than that of the primer concentration, to ensure that the extension of any give template molecule goes to completion. It is reasoned that as template concentration decreased below that of the particle-bound primer, steric effects might become less pronounced due to greater spacing between hybridized strands on the particles. FIG. 2 shows the effect of primer:template concentration ratio on hybridization to particle-bound C₆P12. The coverage of hybridized template is much lower for excess primer (p:t 5:1 and 10:1) as compared to the excess template experiments. In addition, the difference between T12 and T88 hybridization is more pronounced under excess template conditions. However, the data more closely approach the line for optimal hybridization. Note that with limiting the template concentration, it is no longer possible for every primer to bind a complementary (template) strand from solution. The maximum percentage of primers that could bind template at a 5:1 primer:template ratio is 20%. To account for this, hybridization efficiency has been calculated based on 100% hybridization of the template for this and all experiments in which the template concentration is limiting. For T12, the percent occupancy of primers is close to 15% with a 5-fold excess of primer, and close to 9% with a 10fold excess. This corresponds to a hybridization efficiency for T12 of 76% and 88%, respectively. At a ten-fold excess of particle-bound primer, the hybridization efficiency for T12 is largely independent of primer coverage, indicating the decreased importance of steric effects under these conditions.

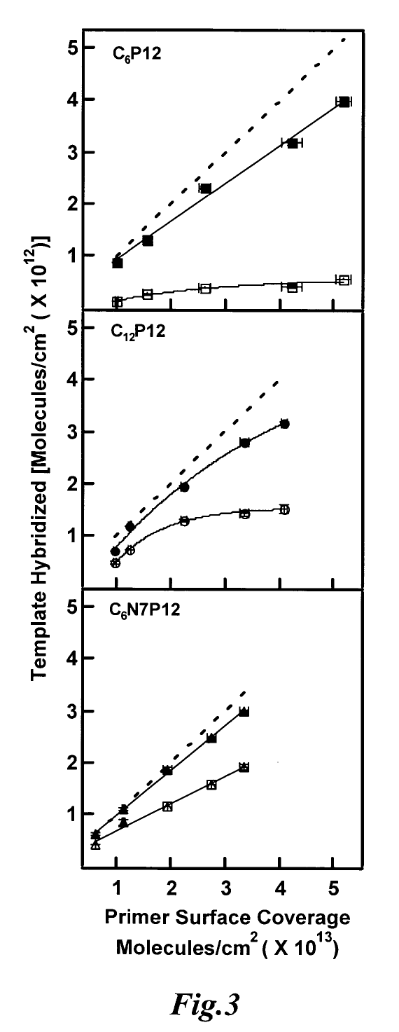

The effect of linker length on hybridization efficiency at a primer:template ratio of 10:1 is shown in FIG. 3. Again, the longer template sequence invariably leads to a lower number of hybridization events. However, the difference in hybridization efficiency between T12 and T88 is linker-dependent, and decreases substantially with increasing linker length. For the intermediate-length linker, C₁₂, hybridization efficiency is strongly dependent upon primer coverage, with a nearly two-fold difference between T12 and T88 (corresponding to 59% and 94%, respectively). The C₆N7 linker gives efficiencies close to 100% for T12 and 75% for T88. The T88 hybridization data can be fit with a line only for the longest linker (FIG. 3 bottom), illustrating the effect of steric crowding at high primer coverages for C₆P12 and C₁₂P12.

Table II summarizes the hybridization efficiency data from the experiments in FIGS. 2 and 3 (Footnote: note that in all cases efficiency was calculated based on the maximum possible hybridization events in a given reaction. In cases where primer was limiting (excess template), hybridization efficiency is the fraction of primers hybridized, while for limiting template (5:1 and 10:1 primer:template ratio), hybridization efficiency is the fraction of template hybridized). These data indicate that, while steric factors are significant for hybridization of solution-phase templates to nanoparticle bound primers, these effects can be greatly diminished by decreasing primer coverage and increasing the distance between the primer sequence and the particle surface. For the short solution phase complement, hybridization efficiencies approach 100% with the long linkers at low primer coverages and low primer:template ratio. While hybridization efficiencies for the long solution phase complement did not reach 100% under these reaction conditions, the importance of linker length, primer coverage and the ratio of surface bound to solution-phase oligonucleotides has been demonstrated. In our experiments, the reaction was allowed to proceed for 2 hours. This was long enough for complete hybridization between particle-bound primers and T12. However, reaction of the longer template, T88, may not have gone to completion. It is expected that, at longer times, greater hybridization efficiencies could be observed.

Extension of Particle-Bound Primers

The extension reaction requires not only efficient hybridization of template to the particle-bound primer, but also accessibility to the DNA polymerase enzyme (in this case, the 68 kDa Klenow fragment). Thus, the extension reaction might be expected to show greater sensitivity to steric effects than hybridization alone. Additional concerns include potential nonspecific adsorption of the enzyme to primer:Au conjugates, and deleterious effects of reaction conditions on conjugate stability. In particular, the elevated temperature (37° C.) and trace amounts of the reducing agent, dithiothreitol (DTT) present during extension might be expected to destabilize the thiol-Au attachment chemistry. To determine the effect of these reaction conditions, conjugates were exposed to various concentrations of DTT at room temperature and at 37° C. No detrimental effects under the extension reaction conditions were observed.

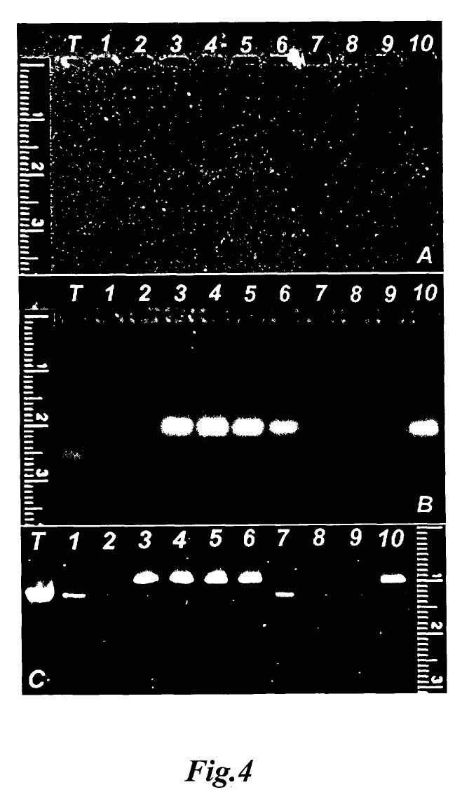

To test for nonspecific adsorption and/or deactivation of Klenow, “spectator” N18:Au or BSA:Au conjugates were added during solution-phase extension of free primer (P12). Extension was determined via fluorescence of incorporated Alexa Fluor® 488-5-dUTP and gel electrophoresis of the extension products. FIG. 4 shows a nondenaturing agarose gel before (A) and after (B) staining with ethidium bromide (EtBr). Fluorescence of the incorporated dUTP is observed at −2 cm in lanes 3-6 and 10. These bands correspond to the dsDNA product of the extended primer-template complex. Following staining with EtBr, contrast is much improved and all of the DNA can be imaged (FIG. 4B). The double-stranded extension product is now clearly visible for lanes 3-6 and 10. Lane 6 (20% N18:Au) in particular appears to have a lower intensity than the particle-free control (lane 3), indicating a lower extension efficiency. Bands at ˜2.6 cm (lanes 1, 7, and 9) correspond to single-stranded template (run in lane T), indicating that no extension occurred in those reactions. The absence of the ˜2.6 cm band in lanes 2 and 8 is expected as no template was added to these reactions.

To determine whether the extension reactions were going to completion (i.e. once the polymerase reaction starts on a give primer-template complex, it copies the full length of that template), a polyacrylamide denaturing gel was run of the same reactions (FIG. 4C). In this case, it is expected to see at least two bands in each of the reactions 3-6 and 10, one for the extended product (copied template strand) and one for the template. This was observed, the band at 1 cm represent the extended primer while the band at 1.3 cm corresponds to the template (the copied template strand runs higher due to the incorporation of Alex dUTP). The observation of only two bands indicates that the extension reaction is going to completion. Incomplete extension would give rise to multiple bands for the various partially extended products. Unexpectedly, a large number of bands were observed in lane 9 (the nonspecific control). These bands appear to result from the partial annealing of two template molecules, with one acting as the “primer” for the other. This would explain the low amount of fluorescent dUTP incorporation shown in Table III. The probability that two template molecules would anneal in the presence of the specific primer is low, since the primer is used in ten-fold excess and the primer-template complex is the energetically favored reaction. Indeed, these additional bands are not observed for reactions that contain P12. Taken together, these gels indicate that extension is occurring and going to completion despite the presence of “spectator” DNA:Au conjugates.

To address the overall efficiency of extension (i.e. the percentage of template molecules that are copied), quantitative data for fluorescent nucleotide incorporation were acquired. A significant (30-40%) decrease is found in the number of fluorescent dUTPs incorporated when “spectator” nanoparticle conjugates are present (Table III). This decreased efficiency could result from nonspecific adsorption of template, enzyme or extended dsDNA onto the N18:Au and BSA:Au particles or from loss of material during column purification. Although primer coverage was not expected to significantly affect nonspecific adsorption to DNA:Au conjugates, this control experiment was run for all three coverages used in the extension experiments because the concentration of Au particles in the reaction was higher for the lower coverage particles (to maintain a costant primer concentration and template concentration for ease of reaction comparison in gels). Thus, at “20% primer” coverage, fivefold more DNA:Au particles are present than at “100%”. Decreased efficiency is observed for extension with this increased concentration of Au particles in solution, although the difference is only ˜13%.

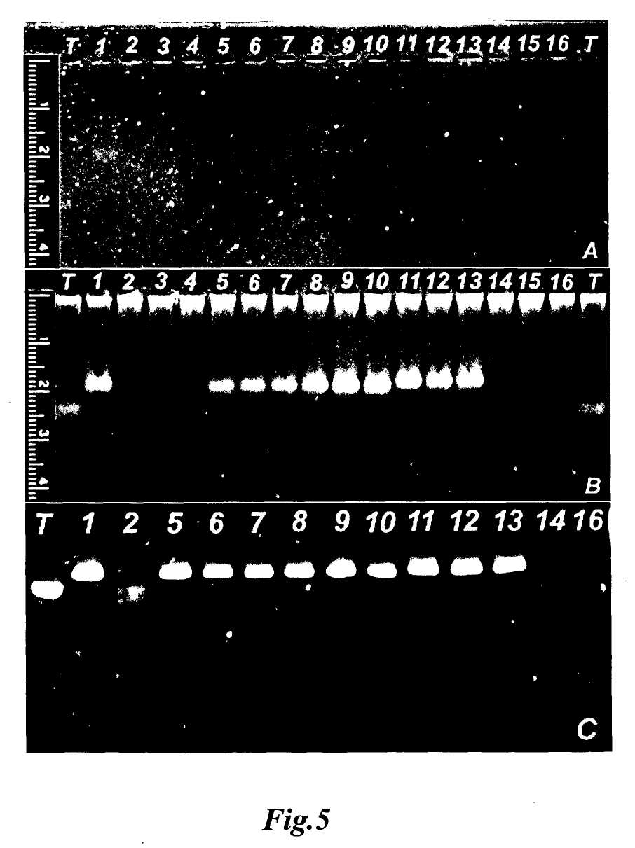

Evidence for extension of particle-bound primers (P12:Au) can be seen in FIGS. 5A and 8, a nondenaturing agarose gel of the extension products run after removal of DNA from the Au nanoparticles. The samples run in each well are described in Table IV. FIG. 5A shows the gel prior to EtBr staining: fluorescence from incorporated nucleotides shows up, albeit weakly, in the wells corresponding to specific primer:Au. After staining with EtBr, contrast is much improved (FIG. 4B). Bands present at −1.9 cm (lanes 1, 5-13) correspond to the double-stranded extension product, while those at ˜2.4 correspond to the template. Thus, extension of the particle-bound primer was successful for all linkers and primer coverages attempted. Note that the bands in lanes 11-13 run slightly higher than the other dsDNA products. This can be explained by the longer linker (C₆N7) used in these reactions. No extension is observed for the noncomplementary controls (lanes 14-16). The brightness of the extended product band for lanes 5-13 (the various particle-bound primers) is not constant. This indicates some difference in efficiency between the different linker and coverage conditions. A denaturing gel was also run for this set of reactions (FIG. 5C). Again, there are two bands present for lanes 1 and 5-13, as expected due to separation of the template from the extended primer strand. Bands corresponding to extended primer are much brighter than those for template, due to fluorescent nucleotide incorporation. There are again at least two bands present in the negative control (lane 2) due to non-specific extension.

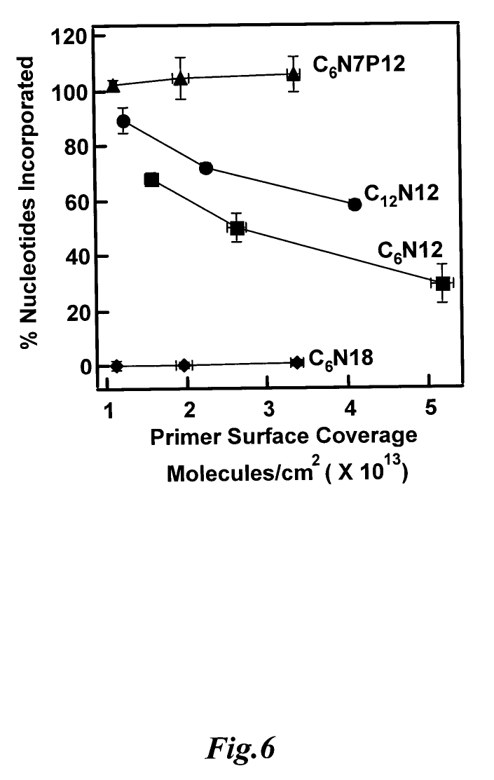

In order to quantitate extension, DNA was removed from Au nanoparticles and fluorescence from incorporated Alexa-dUTPs was measured (Table IV). FIG. 6 shows the results. As could be seen in the gel (FIG. 5B), the primer with longest linker, C₆N7P12, was extended with the greatest efficiency. The C₁₂ and C₆ linkers gave reduced extension efficiencies, in order of their linker lengths. For the C₆P12 and C₁₂P12, surface dilution was critically important for extension. However, for the primer bound through a C₆N7 linker, surface coverage was less important. Note that the low—but detectible—counts observed for the noncomplementary control are due to the template dimerization/extension reaction described above and observable in FIGS. 4C and 5C, and do not correspond to actual primer extension.

The measured extension efficiencies shown in Table IV largely follow the same trends for primer coverage and linker length as observed in the hybridization experiments. For the shorter linkers, extension efficiency is lower and the effect of primer coverage is particularly important. As observed for hybridization, extension is most efficient for low primer coverages. In contrast, for C₆N7, this trend is reversed, with the highest primer coverage yielding the most efficient extension (71%). This result can be understood in light of the data on spectator particles in Table III, which illustrates the detrimental effect of higher particle concentrations on extension efficiency. In order to maintain constant primer and template concentrations as the primer coverage was decreased, more particles were added to the reaction. Thus, the decreased efficiency resulting from greater particle concentrations may be masking the effect of primer coverage on extension efficiency. When this is taken into account by normalizing the extension data to the appropriate control reaction in Table III, it is found that the coverage is unimportant for the C₆N7 linker, and that the extension efficiency of this reaction is 100%. That is, attachment of the primer to the Au particle has had no effect on the incorporation of fluorescent dUTPs as compared to the free primer in the presence of spectator particles. This is unexpected given the maximal hybridization efficiency of 75% observed for this primer:template pair (Table II). This apparent discrepancy can be understood in light of differences in experimental conditions for the hybridization and extension portions of this work. The much higher particle concentrations necessitated by the extension reaction conditions, coupled with the longer reaction time (b/c after hybridization, extension allowed to proceed 2 hours) led to greater hybridization efficiencies than reported in Table II, making possible the unexpectedly high extension efficiencies.

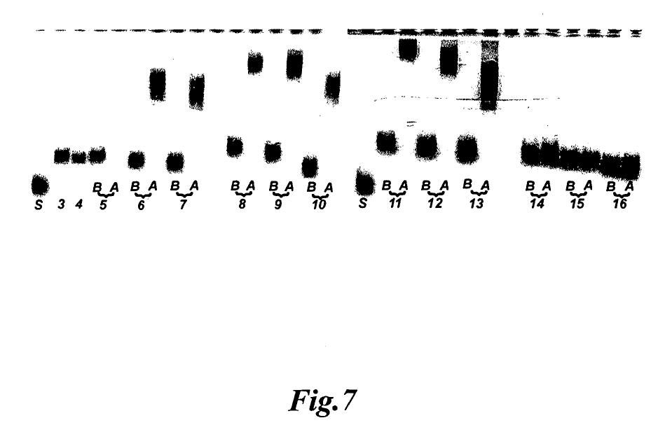

A final experiment was performed to verify that extension occurred while primers were bound to the Au nanoparticles, agarose gels were run of the DNA:Au conjugates themselves. Alivisatos and coworkers recently demonstrated the ability of gel electrophoresis to separate DNA-coated Au nanoparticles based not only on the number of ssDNA molecules attached to each particle but also on the length of the ssDNA (Zanchet et al., Nano Lett. 1:32-5 (2001)). They were able to show separation between DNA:Au conjugates with 50, 80, and 100 base oligomers. FIG. 7 shows an unstained agarose gel of primer:Au conjugates before and after enzymatic extension; bands are visualized by the intense absorbance of the Au particles. Lanes 5-7 contain C₆P12:Au, 8-10 contain C₁₂P12, 11-13 contain C₆N7P12, and 14-16 contain N18:Au, the noncomplementary control. For each set of conjugates, three surface coverages (corresponding to 100%, 50%, and 20% primer solution mole ratio) were run both before (B) and after (A) extension. For all of the complementary primers, a substantial change in electrophoretic mobility is observed upon extension. In all cases the extended conjugates run much slower on the gel, which is consistent with longer DNA bound to the particles. In contrast, no change in band positions was observed for the noncomplementary controls. Note that decreased mobility is not due to aggregation of the Au nanoparticles; all bands are the red color of isolated Au nanoparticles (as opposed to the blue color of aggregates) (Storhoff et al., J. Am. Chem. Soc. 122:4640-50 (2000); Lazarides et al., J. Phys. Chem. B 104:460-7 (2000)) (as a footnote, while the early stages of particle aggregation can give optical absorbances very similar to isolate particles, the conjugates in these experiments have been spun down and resuspended several times; any instability would have resulted in substantial aggregation).

The change in DNA:Au nanoparticle mobility upon extension is greater for the C₆N7P12 and C₁₂P12 primers than for the C₆P12. This is consistent with less efficient extension of the particle-bound C₆P12 primer, as was demonstrated in FIG. 5. Differences between the three primer coverages for any one linker are most apparent for the longer linkers, as the difference between primer and the C₆A6 dilutor molecule is more pronounced.

### CONCLUSIONS

The hybridization of particle-bound oligonucleotide primers has been determined as a function of linker length, surface coverage, the length of the complementary strand in solution and the ratio of particle-bound to free DNA. Extension of Au nanoparticle-bound primers by DNA polymerase has been demonstrated. The efficiency of solution-phase enzymatic extension is decreased 30-40% by the presence of Au particles, presumably due to adsorption of primers, template, and/or enzyme. However, when normalized to this value, the efficiency of extension for particle-bound primers is as high as 100% for the C6N7 linker. Primers with shorter linkers exhibit a strong dependence on primer surface coverage, and in every case result in less nucleotide incorporation.

In summary, the enzymatic processing of metal nanoparticle-bound nucleic acids has been described. It has been found that steric effects remain important, despite the high radius of curvature of the Au nanoparticles used as supports. The factors determined to be important here (linker length, surface coverage) are expected to be generally applicable for all enzymatic reactions on nanoparticle-bound nucleic acids. In addition to extension, it should be possible to, for example, reverse transcribe cDNA onto particles, facilitating gene expression studies, or PCR amplify DNA from Au-bound primers, for subsequent nanoparticle-amplified detection.

TABLE I
Oligonucleotide sequences used in this work

| Abbrev.^{a} | Sequence (5′ to 3′) | Description |
|---|---|---|
| P12 | CGC ATT CAG GAT (SEQ ID NO:1) | Primer |
| N7P12 | taa cat tCG CAT TCA TGA T (SEQ | Primer^{b} |
| | ID NO:2) | |
| AG | AAA AAA (SEQ ID NO:3) | Dilutor |
| N18 | CGA TAA CGG TCG GTA CGG (SEQ ID | Non- |
| | NO:4) | complementary |
| | | primer |
| Tl2 | ATC CTG AAT GCG (SEQ ID NO:5) | First 12 |
| | | nucleotides of |
| | | template |
| N12 | TCT CAA CTC GTA (SEQ ID NO:6) | Non- |
| | | complementary |
| | | “template” |
| T88 | TAC GAG TTG AGA ACA CAG ACG | Template |
| | TAC TAT CAT TGA CGC ATC AGA | |
| | CAA CGT GCG TCA AAA ATT ACG | |
| | TGC GGA AGG AGT TAT CCT GAA | |
| | TGC G (SEQ ID NO:7) | |

^{a}Cₓ before the sequence abbreviation (e.g., C₆P12) denotes the number of CH₂ moieties between the sulfhydryl group and the first nucleotide (i.e. H5C₆H₁₂P12) . F added to any of these sequences denotes the presence of a fluorescein moiety (6-FAM)
^{b}Nucleotides added to the 5′ end of the primer sequence to increase linker length are shown in lower-case letters.

TABLE II
Efficiencies for template hybridization to
particle-bound primers

| | |
|---|---|
| Linker,^{a} Primer: | Hybridization Efficiency^{b,c} |

| template Ratio | T12^{d} | T88 |
|---|---|---|
| C₆, excess template | 20-33% | 11-22% |
| C₆, 5:1 | 55-75% | 17-29% |
| C₆, 10:1 | 76-88% | 10-17% |
| C₁₂, 10:1 | 70-94% | 37-59% |
| C₆N7, 10:1 | 89-98% | 56-75% |

^{a}All primers were P12. See Table I for DNA sequences.
^{b}Hybridization efficiencies are calculated based on the concentration of the limiting DNA strand (for excess template. hybr. eff. = hybridized primers/total primer strands, while for limiting template, hybr. eff. = hybridized templates/total templates.
^{c}Control reactions in which the noncomplementary template, N18, was used in place of P12, calculated hybridization efficiencies were typically undetectable, and in all cases less than 2%.
^{d}Hybridization efficiencies are calculated from the data in FIGS. 2 and 3. Because hybridization efficiency is dependent upon primer coverage, a range of efficiencies are given here for each experiment; in all cases. the low end of the range corresponds to high primer coverage and the high end to lower primer coverage.

TABLE III
Control reactions for this primer:template system

| | | % | | | | |
|---|---|---|---|---|---|---|
| | | Spectator | | | | % Template |
| | | Primer | Enzyme | Template | Nucleotides | Nucleotides |
| Rxn | Sample | on Au^{a} | Present | Present | Incorporated^{b} | Copied^{c} |
| 1 | P12 | N/A | − | + | 0 | 0 |
| 2 | P12 | N/A | + | − | 0 | 0 |
| 3 | P12 | N/A | + | + | 1.19 + 0.05 × | 96.0 ± 4.0 |
| | | | | | 10¹⁴ | |
| 4 | P12 + | 100 | + | + | 8.33 ± 0.13 × | 67.3 ± 0.4 |
| | C₆N18:Au | | | | 10¹³ | |
| 5 | P12 + | 50 | + | + | 7.59 ± 0.05 × | 61.3 ± 0.4 |
| | C₆N18:Au | | | | 1O¹³ | |
| 6 | P12 + | 20 | + | + | 7.28 ± 0.24 × | 58.8 ± 1.9 |
| | C₆N18:Au | | | | 10¹³ | |
| 7 | N18 | N/A | − | + | 0 | 0 |
| 8 | N18 | N/A | + | − | 0 | 0 |
| 9 | N18 | N/A | + | + | 4.26 ± 0.7 × | 0.3 ± 0.1 |
| | | | | | 10¹¹ | |
| 10 | P12 + | N/A | + | + | 8.29 ± 0.19 × | 67.0 ± 1.6 |
| | BSA:Au | | | | 10¹³ | |

Reactions 1, 2, 7 and 8 were negative controls used to determine background counts for fluorescence quantitation. Reactions 1 and 2 contained primer 1 (P12) noted in Table I, while reactions 7 and 8 contained a non-complementary primer (N18). Reactions 4-6 were performed to determine the efficiency of extension in the presence of increasing amounts of colloidal Au present in the reaction, as this will be necessary to keep the primer to template ratio equal for future experiments. Conjugates used in these reactions were made using the N18.

^{a}The % spectator primer on Au refers to the molar ratio of the primer to the diluent at the initial time of conjugate preparation and is close to the primer/diluent ratio of the final product since the primer vs. diluent cover is nearly linear as shown in FIG. 1.

^{b}The amount of nucleotides incorporated was calculated based on the amount of incorporated Alexa dUTP which was determined from a standard curve.

^{c}The % of template nucleotides copied was calculated based on the moles of nucleotides and the moles of template added to each reaction. The values listed for the % copied are normalized to the results of reaction 3.

TABLE IV
Quantitation of Enzymatic Extension from Au-Bound Primers

| | | % | | | | % Template |
|---|---|---|---|---|---|---|
| | | | Enzyme | Template | Nucleotides | Nucleotides |
| Rxn | Sample | Primer on Au^{a} | Present | Present | Incorporated^{b} | Copied^{c} |
| 1 | P12 | N/A | + | + | 6.21 ± 0.02 × | 96.7 ± 3.3 |
| | | | | | 10¹³ | |
| 2 | N18 | N/A | + | + | 2.46 ± 0.64 × | 7.4 ± 1.4 |
| | | | | | 10¹² | |
| 3 | C₆P12:Au | 100 | − | + | 0 | 0 |
| 4 | C₆P12:Au | 100 | + | − | 0 | 0 |
| 5 | C₆P12:Au | 100 | + | + | 1.25 ± 0.28 × | 19.6 ± 4.5 |
| | | | | | 10¹³ | |
| 6 | C₆P12:Au | 50 | + | + | 1.90 ± 0.13 × | 30.7 ± 3.2 |
| | | | | | 10¹³ | |
| 7 | C₆P12:Au | 20 | + | + | 2.41 ± 0.08 × | 40.0 ± 1.4 |
| | | | | | 10¹³ | |
| 8 | C₁₂P12:Au | 100 | + | + | 2.43 ± 0.09 × | 39.3 ± 0.7 |
| | | | | | 10¹³ | |
| 9 | C₁₂P12:Au | 50 | + | + | 2.72 ± 0.05 × | 44.3 ± 1.1 |
| | | | | | 10¹³ | |
| 10 | C₁₂P12:Au | 20 | + | + | 3.29 ± 0.17 × | 52.5 ± 27 |
| | | | | | 10¹³ | |
| 11 | C₆N7P12:Au | 100 | + | + | 4.56 ± 0.15 × | 70.9 ± 41 |
| | | | | | 10¹³ | |
| 12 | C₆N7P12:Au | 50 | + | + | 4.03 ± 0.30 × | 63.9 ± 47 |
| | | | | | 10¹³ | |
| 13 | C₆N7P12:Au | 20 | + | + | 3.76 ± 0.06 × | 59.9 ± 1.2 |
| | | | | | 10¹³ | |
| 14 | C₆N18:Au | 100 | + | + | 3.06 ± 1.84 × | 0.4 |
| | | | | | 10¹³ | |
| 15 | C₆N18:Au | 50 | + | + | 0 | 0 |
| 16 | C₆N18:Au | 20 | + | + | 0 | 0 |

DNA extension comparing the enzymatic efficiency of particle-bound primers to free primers as well as the effect of spacer length between the primer and the gold particle, and localized concentration of primer on the gold particle, on enzymatic efficiency. Extension was achieved using T88 as the template and Klenow for enzymatic extension for 2 hours at 37° C. Quantitation of incorporated nucleotides was determined via Alexa Fluor® 488-5-dUTP using a fluorimeter.

^{a}The % primer on Au refers to the molar ratio of primer to diluent at the initial time of conjugate preparation.

^{b}The amount of nucleotides incorporated was calculated based on the amount of incorporated Alexa dUTP which was determined from a standard curve.

^{c}The % of template nucleotides copied was calculated based on the moles of nucleotides incorporated and the moles of template molecules added to each reaction. The values listed for the % copied are normalized to that obtained for reaction 1.

The disclosure of every patent and publication referred to herein is incorporated by reference in its entirety.

It should be noted that the foregoing description is only illustrative of the invention. Various alternatives and modifications can be devised by those skilled in the art without departing from the invention. Accordingly, the present invention is intended to embrace all such alternatives, modifications, and variances that fall within the scope of the disclosed invention.

What is claimed is:

1. A method for extending a nucleic acid bound to a nanoparticle, the method comprising:
binding to a nanoparticle a single-stranded DNA primer;
annealing to the nanoparticle-bound primer a single-stranded DNA; and
enzymatically extending the primer, thereby extending a nucleic acid bound to a nanoparticle.

2. The method of claim 1, wherein the nanoparticle comprises one or more metals.

3. The method of claim 2, wherein the one or more metals is selected from gold, silver, copper, nickel, rhodium, palladium, and platinum.

4. The method of claim 1, wherein the primer is bound to the nanoparticle via a 5′ thiol linker.

5. A method for reverse transcribing mRNA directly onto a nanoparticle, the method comprising:
binding to a nanoparticle a single-stranded DNA primer;
annealing to the nanoparticle-bound primer a single-stranded mRNA; and
reverse transcribing the mRNA, thereby reverse transcribing mRNA directly onto a nanoparticle.

6. The method of claim 5, wherein the primer is a poly-dT primer.

7. The method of claim 5, wherein the nanoparticle comprises one or more metals.

8. The method of claim 7, wherein the one or more metals is selected from gold, silver, copper, nickel, rhodium, palladium, and platinum.

9. The method of claim 5, wherein the primer is bound to the nanoparticle via a 5′ thiol linker.

10. A method for determining the identity of a specific nucleotide at a defined site in a nucleic acid, the method comprising:
binding to a nanoparticle a single-stranded DNA primer via its 5′ end;
annealing to the nanoparticle-bound primer a single-stranded DNA having a specific nucleotide whose identity is to be determined such that the 3′ end of the primer anneals to a nucleotide flanking the specific nucleotide whose identity is to be determined;
subjecting the nanoparticle-bound primer and annealed DNA to a polymerizing agent in a mixture containing each of ddATP, ddGTP, ddCTP, and ddTTP, wherein each of ddATP, ddGTP, ddCTP, and ddTTP are labeled with a different label, such that the primer is extended by a single nucleotide; and
detecting the identity of the single nucleotide added to the 3′ end of the primer, thereby determining the identity of a specific nucleotide at a defined site in a nucleic acid.

11. The method of claim 10, wherein the nanoparticle comprises one or more metals.

12. The method of claim 11, wherein the one or more metals is selected from gold, silver, copper, nickel, rhodium, palladium, and platinum.

13. The method of claim 10, wherein the primer is bound to the nanoparticle via a 5′ thiol linker.

14. The method of claim 10, wherein the polymerizing agent is a DNA polymerase.

15. A method for introducing sidedness to a metal particle, the method comprising:
binding to a nanoparticle a plurality of first single-stranded DNA molecules;
binding to a solid support a plurality of second single-stranded DNA molecules, wherein the first and second single-stranded DNA molecules are complementary to each other;
contacting the nanoparticle with the solid support such that those first single-stranded DNA molecules nearest the solid support anneal to the second single-stranded DNA molecules contained thereon, and those first single-stranded DNA molecules furthest from the solid support do not anneal to the second single-stranded DNA molecules contained thereon and thus remain free, resulting in a nanoparticle having first single-stranded DNA molecules that are unannealed and free, and first single-stranded DNA molecules that are annealed and not free;
subjecting the nanoparticle to an agent that modifies those first single-stranded DNA molecules that are unannealed and free, but does not modify those first single-stranded DNA molecules that are annealed and not free; and
separating the nanoparticle from the solid support, thereby resulting in a nanoparticle having first and second sides, wherein the first side contains modified first single-stranded DNA molecules, and wherein the second side contains unmodified first single-stranded DNA molecules, thereby introducing sidedness to a nanoparticle.

16. The method of claim 15, wherein the nanoparticle comprises one or more metals.

17. The method of claim 16, wherein the one or more metals is selected from gold, silver, copper, nickel, rhodium, palladium, and platinum.

18. The method of claim 15, wherein each of the first single-stranded DNA molecules are bound to the nanoparticle via a thiol linker.

19. The method of claim 15, wherein the agent is an enzyme.

20. A method for generating covalently immobilized DNA, the method comprising:
binding a first single-stranded DNA primer to a nanoparticle;
mixing the nanoparticle with a DNA having first and second complementary strands under conditions such that the first complementary strand of the DNA anneals to the nanoparticle-bound primer; and
enzymatically extending the first primer, thereby generating covalently immobilized DNA.

21. The method of claim 20, wherein in the mixing step, a second single-stranded DNA primer is mixed with the nanoparticle and the DNA under conditions such that the second complementary strand of the DNA anneals to the second primer, and wherein in the extending step, the second primer is enzymatically extended.

22. The method of claim 21, wherein the mixing and extending steps are repeated one or more times.

23. The method of claim 20, wherein the nanoparticle comprises one or more metals.

24. The method of claim 23, wherein the one or more metals is selected from the group consisting of gold, silver, copper, nickel, rhodium, palladium, and platinum.

25. The method of claim 20, wherein the first single-stranded DNA primer is bound to the nanoparticle via a 5′ thiol linker.


SEQUENCE LISTING (7 sequences)

SEQ ID NO: 1 - DNA, 12 bp, Artificial
  misc_feature: Artificial primer sequence
cgcattcaggat

SEQ ID NO: 2 - DNA, 19 bp, Artificial
  misc_feature: Artificial primer sequence
taacattcgcattcatgat

SEQ ID NO: 3 - DNA, 6 bp, Artificial
  misc_feature: Artificial diluter sequence
aaaaaa

SEQ ID NO: 4 - DNA, 18 bp, Artificial
  misc_feature: Artificial primer sequence
cgataacggtcggtacgg

SEQ ID NO: 5 - DNA, 12 bp, Artificial
  misc_feature: Artificial template sequence
atcctgaatgcg

SEQ ID NO: 6 - DNA, 12 bp, Artificial
  misc_feature: Artificial template sequence
tctcaactcgta

SEQ ID NO: 7 - DNA, 88 bp, Artificial
  misc_feature: Artificial template sequence
tacgagttgagaacacagacgtactatcattgacgcatcagacaacgtgcgtcaaaaattacgtgcggaaggagttatcctgaatgcg